(19) United States
(12) Patent Application Publication
(10) Pub. No.: US 2006/0088909 A1
(43) Pub. Date: Apr. 27, 2006

(54) Virus-like particles, methods of preparation, and immunogenic compositions

(76) Inventors: Richard W Compans, Atlanta, GA (US); Chinglai Yang, Decatur, GA (US); Qizhi Yao, Houston, TX (US); Sang-Moo Kang, Norcross, GA (US)

Correspondence Address: Christopher B Linder;Thomas Kayden Horstemeyer & Risley, Suite 1750, 100 Galleria Parkway NW, Atlanta, GA, 30339-5948, US

(21) Appl. No.: 10/514,462

(22) PCT Filed: May 19, 2003
(86) PCT No.: PCT/US03/15930

§ 371 Date: Nov. 12, 2004

Related U.S. Application Data
(60) Provisional application No. 60/381,557, filed on May 17, 2002.
(60) Provisional application No. 60/454,115, filed on Mar. 11, 2003.
(60) Provisional application No. 60/454,139, filed on Mar. 11, 2003.
(60) Provisional application No. 60/454,584, filed on Mar. 14, 2003.

(51) Int. Cl. C12Q 1/68; C12P 21/06

(52) U.S. Cl. 435/69.1; 435/6

(57) ABSTRACT

Briefly described, virus-like particles, methods of preparing virus-like particles, immunogenic compositions that include virus-like particles, and methods of liciting an immune response using immunogenic compositions that include virus-like particles are described herein. A virus-like particle (VLP) can include a viral core protein that can self assemble into the VLP core and at least one viral surface envelope glycoprotein expressed on the surface of the VLP. The viral protein and the viral surface envelope glycoprotein are from different viruses. Another VLP can include a viral core protein that can self assemble into a VLP core; at least one viral surface envelope glycoprotein expressed on the surface of the VLP; and at least one adjuvant molecule expressed on the surface of the VLP.

### CROSS-REFERENCE TO RELATED APPLICATION

This application claims priority to copending U.S. provisional application entitled, “Chimeric Virus-Like Particles as Vaccine Antigens,” having Ser. No. 60/381,557, filed May 17, 2002; copending U.S. provisional application entitled, “Rift Valley Fever Virus-Like Particle Vaccine,” having Ser. No. 60/454,115 filed Mar. 11, 2003; copending U.S. provisional application entitled, “Targeting HIV Virus-Like Particles To Dendritic Cells,” having Ser. No. 60/454,139, filed Mar. 11, 2003; copending U.S. provisional application entitled, “Rift Valley Fever Virus-Like Particle Vaccine,” having Ser. No. 60/454,584, filed Mar. 14, 2003; copending U.S. provisional application entitled, “Ebola Virus Virus-Like Particles,” having Ser. No. XXXXXXX, filed May 6, 2003; and copending U.S. provisional application entitled, “Stabilizing Virus Envelope Protein Conformation for Inducing Neutralizing Antibody Responses by Treatment with Peptide Corresponding to the Helical Regions (also termed Heptad Repeat Regions) of the Viral Envelope Protein,” having Ser. No. XXXXXXX, filed on May 16, 2003; of which are entirely incorporated herein by reference.

### STATEMENT REGARDING FEDERALLY SPONSORED RESEARCH OR DEVELOPMENT

The U.S. government may have a paid-up license in part of this invention and the right in limited circumstances to require the patent owner to license others on reasonable terms as provided for by the terms of 1R21 AI53514, 1R21 AI44409, and 5R21 AI049116, which all were awarded by the National Institute of Health.

### TECHNICAL FIELD

The present invention is generally related to virus-like particles, methods of preparing virus-like particles, immunogenic compositions that include virus-like particles, and methods of eliciting an immune response using immunogenic compositions that include virus-like particles.

### BACKGROUND

Virus-like particles (VLPs) closely resemble mature virions, but they do not contain viral genomic material (i.e., viral genomic RNA). Therefore, VLPs are nonreplicative in nature, which make them safe for administration in the form of an immunogenic composition (e.g., vaccine). In addition, VLPs can express envelope glycoproteins on the surface of the VLP, which is the most physiological configuration. Moreover, since VLPs resemble intact virions and are multivalent particulate structures, VLPs may be more effective in inducing neutralizing antibodies to the envelope glycoprotein than soluble envelope antigens. Further, VLPs can be administered repeatedly to vaccinated hosts, unlike many recombinant vaccine approaches. An example of a VLP vaccine is the baculovirus-derived recombinant human papillomavirus type (HPV-16) L1 VLP, which was manufactured by Novavax, Inc.

Therefore, VLPs can be used to overcome previous attempts to create vaccines for various viruses such as human immunodeficiency virus (HIV), Ebola virus, severe acute respiratory syndrome (SARS), coronavirus, and Rift Valley Fever virus (RVFV).

### SUMMARY

Briefly described, embodiments of the present invention include novel types of virus-like particles, methods of preparing virus-like particles, immunogenic compositions that include virus-like particles, and methods of eliciting an immune response using immunogenic compositions that include virus-like particles. One exemplary embodiment of a novel type of virus-like particle includes virus-like particle (VLP) that include a viral core protein that can self-assemble into the VLP core and at least one viral surface envelope glycoprotein expressed on the surface of the VLP. The viral protein and the viral surface envelope glycoprotein are from different viruses. Another exemplary embodiment a VLP includes a VLP having a viral core protein that can self assemble into a VLP core; at least one viral surface envelope glycoprotein expressed on the surface of the VLP; and at least one adjuvant molecule expressed on the surface of the VLP.

Another representative embodiment of the present invention includes an immunogenic composition. The immunogenic composition includes a pharmacologically acceptable carrier and at least one of the VLPs described above. Further, another representative embodiment of the present invention includes a method of generating an immunological response in a host by administering an effective amount of one or more of the immunogenic compositions described above to the host. Furthermore, another representative embodiment of the present invention includes a method of treating a condition by administering to a host in need of treatment an effective amount of one or more of the immunogenic compositions described above.

Still another representative embodiment of the present invention includes methods of determining exposure of a host to a virus. An exemplary method, among others, includes the steps of: contacting a biological fluid of the host with one or more of the VLPs discussed above, wherein the VLP is of the same virus type to which exposure is being determined, under conditions which are permissive for binding of antibodies in the biological fluid with the VLP; and detecting binding of antibodies within the biological fluid with the VLP, whereby exposure of the host to the virus is determined by the detection of antibodies bound to the VLP.

Still another representative embodiment of the present invention includes methods of making VLPs. An exemplary method, among others, includes the steps of: providing a viral core protein expression vector; providing a viral surface envelope surface glycoprotein expression vector; providing a adjuvant molecule expression vector; and introducing into a cell the viral core protein expression vector, the viral surface envelope surface glycoprotein expression vector, and the adjuvant molecule expression vector and allowing for expression of the viral surface envelope surface glycoprotein and the adjuvant molecule, whereby the VLP is formed by the cells.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 illustrates a representative virus-like particle (VLP).

FIG. 2 illustrates some representative structural changes that can be made to a representative viral surface envelope glycoprotein.

FIG. 3 illustrates some representative structural changes that can be made to another representative viral surface envelope glycoprotein.

FIGS. 4A and 4B illustrate western blots of representative VLPs incorporating HIV envelope glycoproteins into SHIV VLPs (FIG. 4A) and influenza HA adjuvant molecules into SHIV VLPs (FIG. 4B).

FIGS. 5A through 5B illustrate graphs measuring various characteristics of a number of VLPs that were intranasally introduced into mice. In FIGS. 5A through 5D, “V” represents SHIV VLPs, “HA/V” represents HA/SHIV VLPs, “V+CT” represents SHIV VLPs (10 μg)+CT (10 μg), and “N” represents a negative control (PBS). In addition, the number in parentheses indicates the “μg's” of VLPs used for the immunization of the mice.

FIG. 5A illustrates a graph measuring serum IgG levels specific to HIV envelope glycoproteins.

FIG. 5B illustrates a graph measuring splenocytes producing IFN-γ determined by ELISPOT assay.

FIG. 5C illustrates a graph measuring HIV envelope glycoprotein-specific IgA in vaginal wash.

FIG. 5D illustrates a graph measuring HIV envelope glycoprotein-specific IgA in fecal extracts.

### DETAILED DESCRIPTION

Embodiments of the present invention provide for virus-like particles, methods of using the virus-like particles, and methods of making virus-like particles that can be used in immunogenic compositions to treat conditions in a host, and the immunogenic compositions that include virus-like particles. The virus-like particles can be used to enhance immune responses (e.g., antibody production, cytotoxic T cell activity, and cytokine activity). In particular, virus-like particles can act as a prophylactic as a vaccine to prevent viral infections such as those caused by, for example, the human immunodeficiency virus (HIV), the Corona virus, the Ebola virus, the Rift Valley Fever virus, the Hantaan Virus, the Lassa fever virus, and the Flavivirus.

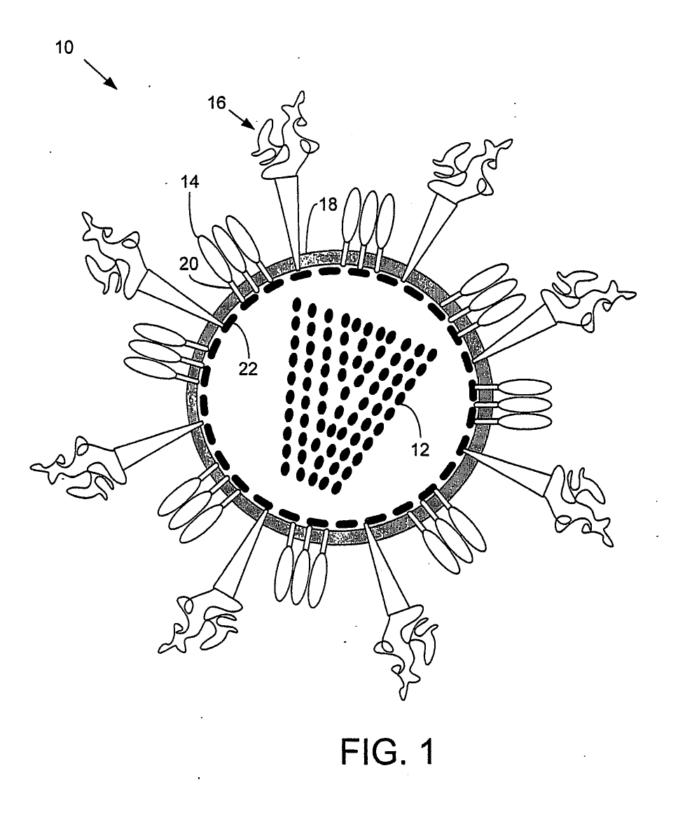

In general, the virus-like particle (“VLP” ) 10 includes at least a viral core protein 12 (hereinafter “viral protein”) and at least one viral surface envelope glycoprotein 14 (e.g., type 1 or type 2 viral surface envelope glycoproteins), as shown in FIG. 1. In addition, the VLP can include at least one adjuvant molecule 16. Furthermore, the VLP can include a lipid membrane 18, viral glycoprotein transmembrane unit 20, and a matrix protein 22. In particular, chimeric VLPs are VLPs having at least one viral surface envelope glycoprotein incorporated into the VLP, wherein the viral core protein and the viral surface envelope glycoprotein are from different viruses. Furthermore, phenotypically mixed VLPs are VLPs having at least one adjuvant molecule incorporated into the VLP wherein the adjuvant molecule are from cells or viruses different than the viral protein.

Viral proteins 12 include proteins that are capable of self-assembling into the VLP (Freed, E. O., J. Virol., 76, 4679-87, (2002)). In particular, the viral proteins 12 can include, but are not limited to, a HIV Gag viral protein (e.g., HIV-1 NL43 Gag (GenBank serial number AAA44987)), simian immunodeficiency virus (SIV) Gag viral protein (e.g., SIVmac239 Gag (GenBank serial number CAA68379)), a murine leukemia virus (MuLV) Gag viral protein (e.g., MuLV Gag (GenBank serial number S70394)), a vesicular stomatis virus (VSV) M viral protein (e.g., VSV Matrix protein (GenBank serial number NP041714)), an Ebola VP40 viral protein (e.g., Ebola virus VP40 (GenBank serial number AAN37506)), a Rift Valley Fever viral N protein (e.g., RVFV N Protein (GenBank serial number NP049344)), a corona M and E virus core protein (GenBank serial number NP040838 for NP protein and NP 040835 for M protein)), a Bunya virus N Protein viral core protein (GenBank serial number AAA47114)), and combinations thereof. It may be necessary to include appropriate surface glycoproteins and/or viral RNA to form the VLP 10.

In general, the viral protein 12 sequence and the corresponding polynucleotide sequence can be found in GenBank and the access numbers can be obtained online at National Center for Biotechnology Information (NCBI). In addition, the sequences identified for the viral proteins 12 above are only illustrative examples of representative viral proteins 12. Furthermore, variants that are substantially homologous to the above referenced viral proteins 12 and viral proteins 12 having conservative substitutions of the above referenced viral proteins 12 can also be incorporated into VLPs 10 of the present invention to enhance the immunogenic characteristics of VLPs.

The viral surface envelope glycoprotein 14, or at least at portion of the viral surface envelope glycoprotein 14, is disposed (e.g., expressed) on the surface of the VLP. The viral surface envelope glycoprotein 14 is disposed on the surface of the VLP so that it can interact with target molecules or cells (e.g., the interaction between the HIV surface envelope glycoprotein and the B cell receptor to activate HIV envelope glycoprotein specific antibody producing B cells) to produce immunogenic responses (e.g., antibody production).

The viral surface envelope glycoproteins 14 can include, but are not limited to, a human immunodeficiency virus (HIV) envelope glycoprotein (e.g., HVSF162 envelope glycoprotein (SEQ ID NO: 1, GenBank serial number M65024)), a simian immunodeficiency virus (SIV) envelope glycoprotein (e.g., SIVmac239 envelope glycoprotein (GenBank serial number M33262)), a simian-human immunodeficiency virus (SHIV) envelope glycoprotein (e.g., SHIV-89.6p envelope glycoprotein (GenBank serial number U89134)), a feline immunodeficiency virus (FIV) envelope glycoprotein (e.g., feline immunodeficiency virus envelope glycoprotein (GenBank serial number L00607)), a feline leukemia virus envelope glycoprotein (e.g., feline leukemia virus envelope glycoprotein (GenBank serial number M12500)), a bovine immunodeficiency virus envelope glycoprotein (e.g., bovine immunodeficiency virus envelope glycoprotein (GenBank serial number NC001413)), a bovine leukemia virus envelope glycoprotein (GenBank serial number AF399703), a equine infectious anemia virus envelope glycoprotein (e.g., equine infectious anemia virus envelope glycoprotein (GenBank serial number NC001450)), a human T-cell leukemia virus envelope glycoprotein (e.g., human T-cell leukemia virus envelope glycoprotein (GenBank serial number AF0033817)), a Rift Valley Fever virus (RVFV) glycoprotein, (e.g., RVFV envelope glycoprotein (SEQ ID NO: 2, GenBank serial number M11157)), a Bunya virus glycoprotein, Lassa fever virus glycoprotein (GenBank serial number AF333969)), Ebola virus glycoprotein (GenBank serial number NC002549)), corona virus glycoprotein (GenBank serial number SARS corona virus spike protein AAP13567)), Arena virus glycoprotein (GenBank serial number AF333969)), Filovirus glycoprotein (GenBank serial number NC002549)), influenza virus glycoprotein (GenBank serial number V01085)), paramyxovirus glycoprotein (GenBank serial number NC002728 for Nipah virus F and G proteins)), rhabdovirus glycoprotein (GenBank serial number NP049548)), alphavirus glycoprotein (GenBank serial number AAA48370 for VEE), flavivirus glycoprotein (GenBank serial number NC001563 for West Nile virus)), and combinations thereof.

In general, the viral surface envelope glycoprotein 14 sequence and the corresponding polynucleotide sequence can be found in GenBank and the access numbers can be obtained online at NCBI. In addition, the sequences identified for the viral surface envelope glycoproteins 14 above are only illustrative examples of representative viral surface envelope glycoproteins 14. Further, variants that are substantially homologous to the above referenced viral surface envelope glycoproteins 14 and viral surface envelope glycoproteins 14 having conservative substitutions of the above referenced viral surface envelope glycoproteins 14 can also be incorporated into VLPs 10 of the present invention to enhance the immunogenic characteristics of VLPs.

In one embodiment, the HIV envelope glycoprotein can be modified and/or truncated to improve the immunogenic properties of the VLP. In particular, the VLP can be conformationally changed by hydrostatic pressure-induced techniques.

In another embodiment, the HIV envelope glycoprotein can be modified to expose neutralizing epitopes in the HIV envelope glycoprotein by removing obstructing structural features such as, but not limited to, glycosylation sites, the V1 loop, the V2 loop, and the V3 loop. By eliminating these obstructing features, the immunogenic properties of the VLP can be enhanced.

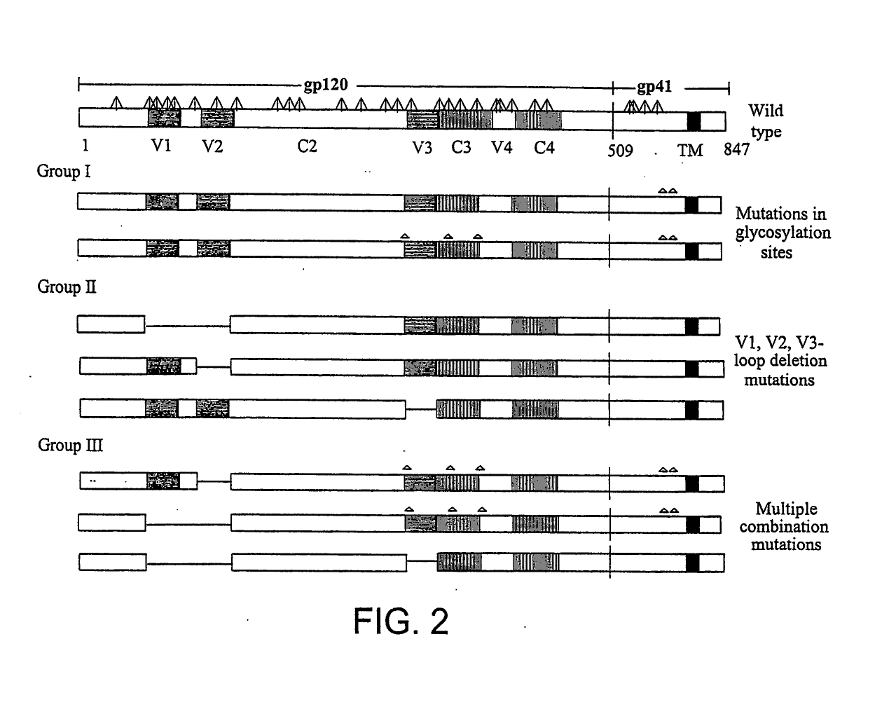

FIG. 2 illustrates some representative structural changes that can be made to the HIV 89.6 envelope glycoprotein (GenBank serial number AAA81043, SEQ ID NO: 3). The arrows in FIG. 2 indicate the N-glycosylation motifs in the HIV 89.9 viral surface envelope glycoprotein as well as the V1 (amino acids 128-164 of SEQ ID NO: 3) loop, V2 (amino acids 164-194 of SEQ ID NO: 3) loop, and V3 (amino acids 298-329 of SEQ ID NO: 3) loop domains. Deletions of the loops is shown by removing the corresponding sequence in the HIV 89.6 envelope glycoprotein shown in FIG. 2. When the loop is deleted, a 3 amino acid linker (GAG) is inserted into the loops former position in the sequence. The glycosylation motifs mutated are denoted by triangles. Three glycosylation motif mutations of Asn to Gln in the V3/C3 (amino acids 298-402 of SEQ ID NO: 3) domains are performed on amino acids 301, 341, and 362. In the gp41 domain (amino acids 509-853 of SEQ ID NO: 3), the glycosylation motif mutations are performed on Asn in amino acid position 623 and 635. TM denotes the transmembrane domain (amino acids 681-7033 of SEQ ID NO: 3). In FIG. 2, Group I illustrates the glycosylation motif mutations in gp41, the V1 loop domains, the V2 loop domain, and the V3-C3 loop domain. Group II illustrates variable loop (V1, V2, and/or V3) deletion mutations, while Group III illustrates representative multiple combination mutations.

For example, mutations of glycosylation sites in gp41 can be performed to enhance the immunogenic properties of a VLP incorporating the HIV envelope glycoprotein. Although most of gp41 appears to be completely occluded in the HIV-1 envelope spike, recent studies indicate that regions of gp41 close to the transmembrane domain are accessible to neutralizing antibodies (Abs). Several mAbs (2F5, Z13, 4E10), which neutralize a broad range of primary HIV-1 isolates, are known to bind to the extracellular domain of gp41. However, attempts to elicit antibodies having these properties by immunization with linear peptide epitopes or with other carrier proteins containing peptide epitopes have not been successful (Coeffier et al., Vaccine, 19, 684-693, (2000); Eckhart, L., et al., J. Gen. Viro., 77 (Pt. 9), 2001-2008, (1996); and Liang, X., et al., Vaccine, 17, 2862-2872, (1999)). These studies indicate that the introduction of neutralizing antibodies against gp41 epitopes may be dependent on the native form of trimeric gp120-gp41 and that these epitopes may not be immunodominant, possibly due to the presence of N-glycans. As shown in FIG. 2, the gp41 domain in the HIV-1 envelope glycoprotein contains four conserved glycosylation motifs (#22 (amino acids 608 of SEQ ID NO: 3), #23 (amino acids 613 of SEQ ID NO: 3), #24 (amino acids 622 of SEQ ID NO: 3), and #25 (amino acids 634 of SEQ ID NO: 3), and viruses with single or double mutations in these glycosylation sites replicated in both human and monkey T cell lines (Johnson, W. E., et al., J. Virol., 75, 11426-11436, (2001)). Removing the glycosylation motifs #24 (amino acid 622 of SEQ ID NO: 3) and #25 (amino acid 634 of SEQ ID NO: 3) near the neutralizing epitopes may increase the exposure of these epitopes and thus, enhance the induction of neutralizing antibodies against the gp41 domain (numbering glycosylation motifs from the N-terminus of the HIV 89.6 envelope glycoprotein).

It should also be note, that upon binding of the HIV envelope glycoprotein to its receptor molecules or the shedding of its surface subunit (gp120), its transmembrane subunit (gp41) converts into a six-helix bundle configuration, which is highly immunogenic but only present non-neutralizing epitopes (Kim, P. S., Annual Reviews of Biochemistry, 70, 777-810, (2001)). The use of a peptide containing the amino acid sequence corresponding to a segment of the gp41 (amino acid 629 to 664 of the SEQ ID NO: 1) can effectively block the transition into the undesirable six-helix bundle configuration. Therefore, treatment of VLPs with such a peptide can help to preserve the HIV envelope glycoprotein on the surface of the VLP to retain its native configuration for more efficient exposure of neutralizing epitopes and thus the induction of neutralizing antibodies. Such structural features are common to the envelope glycoproteins of many viral families, including, but not limited to, the envelope glycoproteins of retrovirus, influenza virus, and parainfluenza virus. Thus, such a VLP treatment approach can be applied to a variety of VLP vaccines.

In another example, the V1 loop, the V2 loop, and the V3 loop can be deleted to enhance the immunogenic properties of VLPs. Deletion of individual V1 or V2 loops does not reduce the potential of the virus to replicate in PBMCs or alter the co-receptor binding of the viral surface envelope glycoprotein (Stamatatos, L., et al., Aids Res. Hum. Retroviruses, 14, 1129-1139, (1998)). HIV-1 mutants lacking the V1 and V2 loops in gp120 exhibited increased sensitivity to neutralization by antibodies directed against V3 and a CD4-induced epitope on gp120, and by sera collected from patients infected with clades B, C, D, and F HIV-1 primary isolates (Cao, J., et al., J. Virol., 71, 9808-9812, (1997) and Stamatatos, L., and C. Cheng-Mayer, J. Virol., 72, 7840-7845, (1998)). These studies suggest that the V2 loop or V2 together with the V1 loop shields some important neutralization epitopes with an overall structure that appears to be conserved among different HIV-1 primary isolates. Thus, deleting the V1-V2 loop or V2 loop may expose hidden neutralizing epitopes.

The V3 loop of the HIV envelope glycoprotein is highly variable and also constitutes a dominant epitope for the antibody response. Although neutralizing antibodies against this region are frequently detected, they are often strain-specific (Sattentau, Q. J., et al., Virol., 206 (1), 713-7, (1995) and D'Souaza, M. P., et al., Aids, 9, 867-874, (1995)). Furthermore, deletion of the V3 loop has also been shown to increase the exposure of epitopes induced by sCD4 binding (Sanders, R. W., et al., J. Virol., 74, 5091-5100, (2000)). Lu et al. (Aids Res. Hum. Retroviruses, 14, 151-155, (1998)) compared antibody induction by gene-gun immunization of rabbits with DNA vectors expressing HIV-1 IIIB Gp160, Gp140, Gp120 and their corresponding V1/V2/V3 triple loop deletion mutants. These results showed that deletion of variable loops induced higher ELISA antibody responses but not neutralizing antibody responses.

In another study, Garrity et al. (J. Immunol., 159, 279-289, (1997)) showed that immunization of guinea pigs using recombinant vaccinia virus followed by protein boosting with mutant viral surface envelope glycoprotein 120, in which glycosylation sites were introduced to mask the immunodominant domain in the V3 loop, was more effective in inducing cross-reactive neutralizing antibodies against a divergent strain of the same subtype. Thus, eliminating the immunodominant epitopes in the V3 loop may enhance induction of cross-reactive antibodies. Furthermore, Kiszka et al. (J. Virol., 76, 4222-4232, (2002)) reported that immunization of mice using DNA vaccines encoding HIV envelope glycoproteins with V3 loop deletions induced broader cellular immune responses to subdominant epitopes and was more effective in conferring protection against challenge with recombinant vaccinia virus expressing heterologous HIV envelope glycoproteins, indicating that deletion of the V3 loop may also be advantageous in inducing broader cellular immune response.

In another embodiment, the RVFV envelope glycoprotein can include, but is not limited to, a RVFV GC envelope glycoprotein (SEQ ID NO: 4) and a RVFV GN envelope glycoprotein (SEQ ID NO: 5). The RVFV GC and GN envelope glycoproteins can be modified to enhance the immunogenic properties of the VLP 10. For example, the RVFV GC and GN envelope glycoproteins can be modified by truncating the cytoplasmic domain for the RVFV GC (amino acids 492-507 of SEQ ID NO: 4) and GN envelope glycoproteins (amino acids 458-527 of SEQ ID NO: 5).

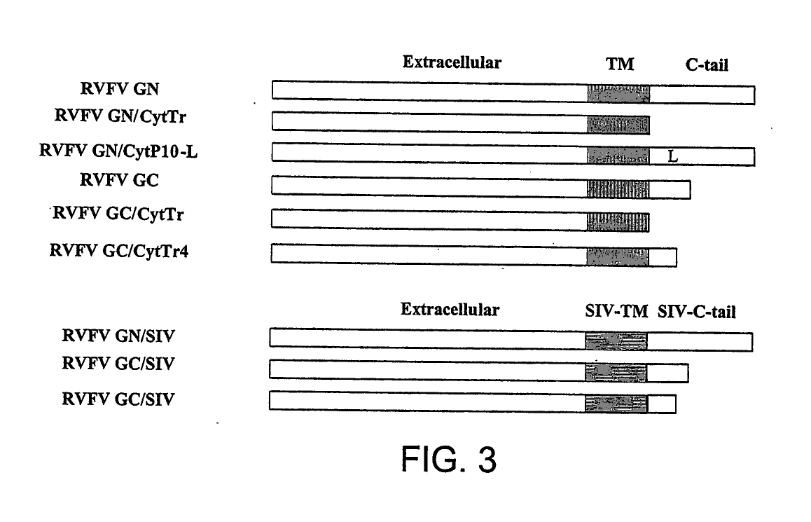

FIG. 3 illustrates some representative structural changes that can be made to the RVFV GN and GC envelope glycoproteins. For example, the RVFV GC and GN envelope glycoproteins can be modified by truncating the cytoplasmic domain for the RVFV GC (amino acids 492-507 of SEQ ID NO: 4) and GN envelope glycoproteins (amino acids 458-527 of SEQ ID NO: 5). In addition, the RVFV GN envelope glycoprotein can mutated by replacing the proline residue (amino acid 537 of SEQ ID NO: 5) from the cytoplasmic domain (conserved between RVFV and PTV GN envelope glycoprotein) with a leucine residue. Such modifications should increase levels of surface expression of the RVFV envelope glycoproteins and therefore increase their incorporation into VLPs. Thus, the effectiveness of the VLPs to elicit immune response against RVFV envelope glycoproteins may be enhanced since the VLPs may contain more RVFV envelope glycoproteins per unit amount.

Furthermore, the RVFV GC and GN envelope glycoproteins can be modified by replacing the transmembrane domain and/or the cytoplasmic tails of the RVFV GC and GN envelope glycoproteins with the transmembrane domain and the cytoplasmic tail of the SIV envelope glycoprotein. Studies on retrovirus assembly have shown that efficient incorporation of viral surface envelope glycoproteins may involve specific interaction between viral Gag proteins and the cytoplasmic domain of the viral surface envelope glycoprotein (Cosson, P., et al., EMBO J., 15, 5783-5788, (1996); Vincent, M. J., et al., J. Virol., 73, 8138-44, (1999); and Wyma, D. J., et al., J. Virol., 74, 9381-7, (2000)). Therefore, replacing the transmembrane domain and cytoplasmic tails of RVFV GN and GC envelope glycoproteins with those of the HIV or SIV envelope glycoprotein (SEQ ID NO: 6) or cytoplasmic tails (SEQ ID NO: 8) may produce chimeric proteins with increased cell surface expression and more efficient incorporation into SIV VLPs.

In previous studies, SIV envelope glycoproteins containing this truncated cytoplasmic domain yielded high levels of cell surface expression and more efficient incorporation into SIV VLPs in comparison to the SIV envelope glycoprotein with a full length cytoplasmic domain of over 150 amino acids (Vzorov, A. and Compans, R. W., J. Virol., 74, 8219-25, (2000)). Furthermore, the cytoplasmic domain of the SIV envelope glycoprotein contains a Tyr-based endocytosis signal, which has been shown to induce rapid endocytosis of the SIV envelope glycoprotein and lead to reduced surface expression (Labranche, C. C., et al., J. Virol., 69, 5217-5227, (1995)). Thus, attaching a truncated SIV envelope glycoprotein cytoplasmic domain to the RVFV envelope glycoproteins that are expressed on cell surface may produce enhanced VLPs.

In addition, Tyr residue can be replaced by Cys in the attached SIV cytoplasmic domain sequence (amino acid 16 of SEQ ID NO: 8) to further augment surface expression of designed chimeric proteins. This design for chimeric proteins can be applied to both RVFV GN and GC envelope glycoproteins. Such modifications may increase levels of surface expression of the RVFV envelope glycoproteins and therefore increase their incorporation into VLPs. Thus, the effectiveness of the VLPs to elicit immune response against RVFV envelope glycoproteins may be enhanced, since the VLPs contain more RVFV envelope glycoproteins per unit amount.

The adjuvant molecule 16, or at least a portion of the adjuvant molecule 16, is disposed (e.g., expressed) on the surface of the VLP 10. The adjuvant molecule 16 can interact with other molecules or cells (e.g., mucosal surfaces having sialic acid residues disposed thereon and antigen-presenting cells such as dendritic cells and follicular dendritic cells). The adjuvant molecule 16 can include, but is not limited to, an influenza hemagglutine (HA) adjuvant molecule (GenBank access number J02090), a parainfluenza hemagglutine-neuraminidase (HN) adjuvant molecule (GenBank access number z26523 for human parainfluenza virus type 3 HN sequence information), a Venezuelan equine encephalitis (VEE) adjuvant molecule (GenBank access number nc001449), a fms-like tyrosine kinase ligand (F1t3) adjuvant molecule (GenBank access number NM013520), a C3d adjuvant molecule (GenBank access number nm009778 for mouse C3 sequence and access number nm000064 for human C3 sequence), a mannose receptor adjuvant molecule, and a CD40 adjuvant molecule (GenBank access number m83312 for mouse CD40).

In general, the adjuvant molecule 16 sequence and the corresponding polynucleotide sequence can be found in GenBank and the access numbers can be obtained online at the NCBI. In addition, the sequences identified for the adjuvant molecules 16 above are only illustrative examples of representative adjuvant molecules 16. Further, variants that are substantially homologous to the above referenced adjuvant molecules 16 and adjuvant molecules 16 having conservative substitutions of the above referenced adjuvant molecules 16 can also be incorporated into VLPs 10 of the present invention to enhance the immunogenic characteristics of VLPs.

Mucosal immunity is critical for prevention of infection by aerosolized virus because mucosal cells can neutralize the virus and/or blocking virus attachment of the virus to the mucosal cells with secreted antibodies. However, little success has been documented for eliciting strong mucosal immune responses by non-replicating vaccines against viruses other than influenza, which is attributed, at least in part, to the difficulty of targeting the antigens to mucosal sites. In contrast, inactivated influenza virus has been shown to induce strong mucosal immune responses when administered mucosally, which may be the result of the strong binding affinity of the HA adjuvant molecule for sialic acid residues that are abundant at mucosal surfaces. Therefore, the affinity of HA adjuvant molecule for sialic acids may be utilized for targeting VLPs to mucosal surfaces. As discussed in more detail below, HA/SHIV VLPs are highly effective in eliciting strong mucosal immune responses against SHIV antigens when administered intranasally to mice. Thus, incorporating HA adjuvant molecules into VLPs may enhance the immunogenic properties of VLPs.

The following is a non-limiting illustrative example of an embodiment of the present invention that is described in more detail in (Guo et. al., “Enhancement of Mucosal Immune Responses by Phenotypically Mixed Influenza HA/SHIV Virus-Like Particles”, (2003), in press), which is incorporated herein by reference. This example is not intended to limit the scope of any embodiment of the present invention, but rather is intended to provide specific experimental conditions and results. Therefore, one skilled in the art would understand that many experimental conditions can be modified, but it is intended that these modifications be within the scope of the various embodiments of this invention.

Mucosal immune responses against HIV play an important role in prevention of HIV infection and transmission, as the mucosal surface is the major site for initial HIV infection. Being the first line of defense, mucosal immunity is critical for prevention of infection by neutralizing virus and/or blocking virus attachment with secreted antibodies. However, little success has been documented for eliciting strong mucosal immune responses against HIV, which is attributed at least in part to the difficulty of targeting the antigens to mucosal sites. In contrast, inactivated influenza virus has been shown to induce strong mucosal immune responses when administered mucosally, a likely result of the strong binding affinity of its HA adjuvant molecule for sialic acid residues that are abundant on mucosal surfaces. Therefore, VLPs incorporating the HA adjuvant molecule may be suited to target mucosal surfaces since the HA adjuvant molecule has an affinity for sialic acid.

To enhance mucosal immune responses using SHIV VLPs as a mucosal AIDS vaccine, phenotypically mixed influenza HA/SHIV virus-like particles (HA/SHIV 89.6 VLPs) were produced and used to intranasally immunize C57B/6J mice. Production of phenotypically mixed HA/SHIV 89.6 VLPs, which possess both biologically active HIV envelope glycoproteins and influenza HA adjuvant molecules, may be important in elicitation of enhanced immune responses against HIV envelope proteins. Therefore, baculovirus-derived SHIV 89.6 VLPs and HA/SHIV 89.6 VLPs were produced through co-infection of insect cells with rBV SIV Gag, rBV HIV envelope glycoprotein, both with and without rBV HA adjuvant molecule.

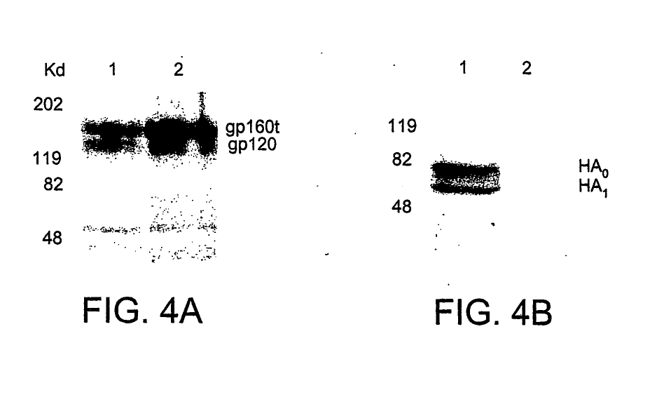

FIGS. 4A and 4B illustrate that both HIV envelope glycoproteins and influenza HA adjuvant molecules can be detected in HA/SHIV 89.6 VLPs by using Western Blot analysis blotting with antibody against HIV envelope glycoproteins (FIG. 4A) or influenza HA adjuvant molecules (FIG. 4B), respectively. Both HIV envelope glycoprotein and influenza HA adjuvant molecule are partially cleaved into their active state gp120 and HA1. In addition, the ability of chimeric VLPs to induce hemagglutination (HA) was examined, a functional property of influenza HA. HA titers of chimeric VLPs were determined by incubating equal volumes of serial two-fold dilutions of HA/SHIV VLPs in PBS-def (PBS deficient in Mg²⁺ and Ca²⁺) with chicken red blood cells (final concentration 0.5%) for 1 hour (h) at room temperature. The HA titer of HA/SHIV 89.6 chimeric VLPs were found to be as high as 1:4000, whereas SHIV 89.6 VLPs showed negative in HA titer.

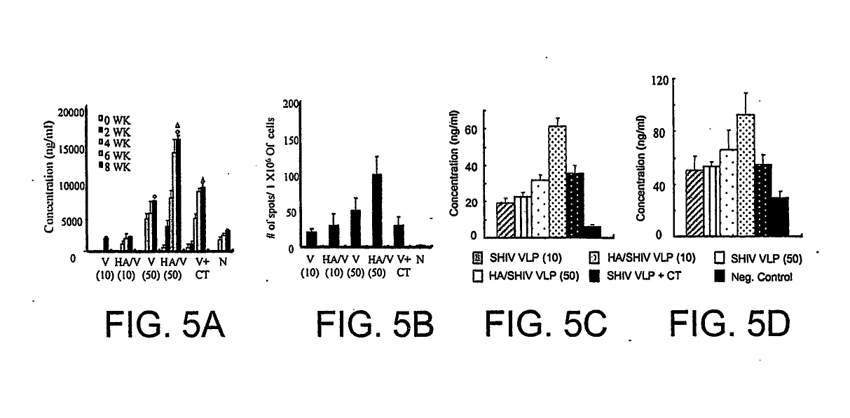

FIGS. 5A through 5B illustrate graphs measuring various characteristics of a number of VLPs that were intranasally introduced into mice. In FIGS. 5A through 5D, “V” represents SHIV VLPs, “HA/V” represents HA/SHIV VLPs, “V+CT” represents SHIV VLPs (10 μg)+cholera toxin (CT) (10 μg), and “N” represents a negative control (PBS). In addition, the number in parentheses indicates the “μg's” of VLPs used for immunization of the mice.

FIG. 5A is a graph measuring serum IgG levels specific to HIV envelope glycoproteins. FIG. 5B is a graph measuring splenocytes producing IFN-γ determined by ELISPOT assay. FIG. 5C is a graph measuring HIV envelope glycoprotein-specific IgA in vaginal wash. FIG. 5D is a graph measuring HIV envelope glycoprotein-specific IgA in fecal extracts.

In particular, systemic and mucosal antibody responses, as well as cytotoxic T cell (CTL) responses, of mice immunized with SHIV 89.6 VLPs or HA/SHIV 89.6 VLPs are shown in FIGS. 5A through 5D. Intranasal immunizations were given with VLPs either with or without addition of CT. The level of serum IgG production to HIV envelope glycoprotein was found to be highest in the group immunized with phenotypically mixed HA/SHIV 89.6 VLPs. Similarly, mucosal IgA production was also found to be enhanced in the group immunized with HA/SHIV VLP mucosally. Analysis of the IgG1/IgG2a ratio indicated that a Th1-oriented immune response resulted from these VLPs immunizations. HA/SHIV VLP-immunized mice also showed significantly higher CTL responses than those observed in SHIV VLP-immunized mice. Furthermore, a MHC class I restricted T cell activation ELISPOT assay showed elevated IFN-γ, IL-2, and IL-12 production in HA/SHIV VLP-immunized mice, indicating that phenotypically mixed HA/SHIV VLPs can enhance both humoral and cellular immune responses at multiple mucosal sites. Thus, a heterologous adjuvant molecule, the HA adjuvant molecule, can be coexpressed with retrovirus proteins in infected cells resulting in its efficient incorporation into retroviral VLPs in a biologically active form. In addition, the resultant VLPs exhibit enhanced immunogenicity, especially when delivered by a mucosal route. These results demonstrate the feasibility of construction of a range of VLPs containing heterologous surface molecules for possible use as improved vaccine antigens. Therefore, chimeric HA-containing VLPs may be used for the mucosal immunization against HIV.

The possibility of pre-existing immunity to the influenza HA protein is a factor that should be considered in the evaluation of HA-VLP vaccines. However, it is uncertain that the existence of such preexisting immunity would negatively impact the immune responses to the VLPs (in marked contrast to immunization using replicating vectors). It is in fact possible that preexisting antibodies would lead to production of immune complexes would enhance targeting of VLPs to follicular dendritic cells and thus result in stimulation of B cell responses.

Nevertheless, alternative approaches can be applied for mucosal targeting of VLPs. One possibility is to utilize influenza HA adjuvant molecules from other influenza virus species to which there is no preexisting immunity in the human population. There are about 15 such non-cross reactive serotypes of HA adjuvant molecules, which have been identified, which are antigenically non-overlapping based on tests with polyclonal immune sera. The 15 non-cross reactive serotypes of HA viruses and their replication are described in the following publications: Lamb, R. A. and Krug, R. M., Orthomyxoviridae, (1996) and Fields, B. N., et. al., Editors, Field's Virology, Lippincott-Raven Publishers, Philadelphia, Pa., 1353-1395, (1996). Thus, VLPs can be produced containing one or more of these alternative HA adjuvant molecule subtypes, therefore avoiding a possible affect of preexisting anti-HA immunity on induction of immune responses against VLP antigens. It should be noted that some influenza HA adjuvant molecules from other species may bind preferentially to sialic acid linkages not found on human cells. This property, however, can be modified by mutation of specific HA amino acids (Vines, A., et al., J. Virol., 72, 7626-7631, (1998)).

An alternative approach to using HA adjuvant molecules is the production of VLPs containing parainfluenza virus HN adjuvant molecules. Like HA adjuvant molecules, the HN adjuvant molecules attach specifically to the sialic acid residues at mucosal surfaces. Thereore, chimeric VLPs containing HN adjuvant molecules should have similar mucosal targeting properties as the HA adjuvant molecules. However, immune responses to the proteins of human parainfluenza viruses are of relatively short duration, and reinfections with the same viral serotypes are known to occur (Glezen, W. P., et al., J. Infect. Dis., 150, 851-857, (1984)). Thus, as compared with HA, it is less likely that preexisting immunity to the HN adjuvant molecules of a parainfluenza virus would affect mucosal delivery of a VLP vaccine.

HN-VLPs may be easier to produce in modified vaccinia Ankara expression systems rather than baculovirus expression systems. This is because the release of HA chimeric VLPs from mammalian cells would require addition of exogenous neuraminidase (Bosch, V., et al., J. Gen. Virol., 82, 2485-2494, (2001)) since sialic acid would be added to the envelope glycoproteins as a terminal sugar and lead to aggregation of VLPs at the cell surface (which does not occur in the insect cell-produced VLPs). In contrast, HN carries its own neuraminidase. Studies of viral pseudotypes have shown that the glycoproteins of parainfluenza viruses including the HN adjuvant molecule can be assembled into virions of retroviruses, indicating that this type II membrane protein can be incorporated into VLPs (Spiegel, M. et al., J. Virol., 72, 5296-5302, (1998))., Thus, incorporating HN adjuvant molecules into VLPs may enhance the immunogenic properties of VLPs.

Antigen presenting cells can be targeted by VLPs by including one or more of the following adjuvant molecules on the surface of the VLP: the VEE adjuvant molecule, the F1t3 adjuvant molecule, the mannose adjuvant molecule, the CD40 adjuvant molecule, and the Cd3 adjuvant molecule. In particular, the VEE adjuvant molecule, the F1t3 adjuvant molecule, the mannose adjuvant molecule, and the CD40 adjuvant molecule can be used to target dendritic cells, while the Cd3 adjuvant molecule can be used to target follicular dendritic cells.

Dendritic cells (DCs) are very efficient antigen presenting cells involved in priming native CD4 and CD8 T cells, thus inducing primary immune responses and permitting establishment of immunological memory (Inaba, K., et al., J. Exp. Med., 166:182-194, (1987) and Inaba, K., et al., J. Exp. Med., 172:631-640, (1990)). Antigens taken up by DCs are expressed at the cell surface in the form of peptides associated with MHC class II, which stimulates CD4 Th cells. For induction of CD8 T cells, MHC class 1 associated peptides are derived from endogenously synthesized proteins as well as from some exogenous antigens (e.g., infectious agents, dying cells, proteins associated with inert particles, and immune complexes) by DC endocytosis (Heath, W. R. and F. R. Carbone, Curr. Opin. Immunol., 11:314-318, (1999); Reimann, J. and R. Schirmbeck, Immunol. Rev., 172; 131-152, (1999); Regnault, A., et al., J. Exp. Med., 189:371-380, (1999); and Machy, P., et al., Eur. J. Immunol., 30:848-857, (2000)). DCs harboring immune complexes also stimulate naive B cells (Wykes, M., J. Immunol., 161, 1313-1319, (1998) and Dubois, B., et al., Biol., 70, 633-641, (2001)). The highly developed Ag-presenting capacity of DCs has led to their study of cellular vaccine adjuvants for the immunotherapy of cancer (Schuler, G. and R. M. Steinman, J. Exp. Med., 1986: 1183-1187, (1997) and Baggers, J., et al., J. Clin. Oncol., 18:3879-3882, (2000)). HIV and SIV virions interact with DCs via DC-SIGN and/or CD4 receptors; however, this interaction appears to preferentially result in infection of the DCs as well as transmission to other target cells rather than potentiation of an immune response (Geijtenbeek, T. B., et al., Cell, 100: 587-597, (2000) and Geijtenbeek, T. B., et al., Immunol. Lett. 79:101-107, (2001)). On the other hand, inert particulate antigens like VLPs are very attractive target for antigen presenting cells, particularly DCs (Bachmann, M. F., et al., Eur. J. Immunol., 26:2595-2600, (1996); Ruedl, C., et al., Eur. J. Immunol., 32:818-825, (2002) and Da Silva, D. M., et al., Int. Immunol., 13:633-641, (2001)). Therefore, the interaction of VLPs with DCs may result in potentiating DCs to initiate T cell activation.

The possible advantage of targeting vaccine antigens to DCs is indicated by the extremely small number of DCs in peripheral tissues and in blood, where DCs represent less than 1% of total cell number. F1t3 ligand (FL) adjuvant molecule (GenBank access number NM013520) is a hematopoietic growth factor that has the unique ability to expand the number of both CD8α- and CD8α+DC subsets (Lyman, S. D., et al., Cell, 75:1157-1167, (1993); Maraskovsky, E, et al., J. Exp. Med., 184:1953-1962, (1996); Maraskovsky, E, et al., Blood, 96:878-884, (2000) and Pulendran, B., et al., J. Immunol., 159:222-2231, (1997)). Such expansion of DCs in mice resulted in dramatic increases in Ag-specific B and T cell responses (Pulendran, B., et al., J. Exp. Med., 188, 2075-2082, (1998)), enhanced T-cell mediated immune responses (Pisarev et al., Int J Immunopharmacol, 11, 865-76, (2000)), and protective immunity to Listeria monocytogenes (Gregory, S. H., et al., Cytokine, 13:202-208, (2001)). It is suggested that FL treatment increases the capacity of DCs as antigen presenting cells by up-regulating MHC and costimulatory molecules (CD40, CD86), and by inducing production of cytokines (IFN-γ, IL-2, IL-12 or IL-4) (Pulendran, B., et al., J. Exp. Med., 188, 2075-2082, (1998) and Pulendran, B., et al., Proc. Natl. Acad. Sci., U.S.A. 96, 1036-1041, (1999)). Therefore, incorporation of FL adjuvant molecules into VLPs may enhance the immunogenic properties of the VLPs.

The VLP can be produced to include the FL adjuvant molecule by PCR-amplifying and cloning the whole mouse FL gene including the signal sequence and transmembrane (TM) domain into rBV transfer vector pc/pS1. To construct a rBV expressing FL, Sf9 insect cells can be co-transfected with Baculogold DNA (available from PharMingen, Inc.) and the pc/pS1 transfer vector containing the FL gene.

The incorporation of the FL adjuvant molecule into VLPs can be enhanced by modifiying the FL adjuvant molecule. In particular, the extracellular coding domain of the FL gene (from the end of signal peptide to the start of the TM segment) (SEQ ID NO: 7) can be fused to the N-terminus of the SIV Env glycoprotein-41 TM domain (SEQ ID NO: 6) and the tPA signal peptide can be fused to the N-terminus of the FL-chimeric protein (SEQ ID NO: 9). An alternative approach is to produce a glycosyl-phosphatidylinositol (GPI)-anchored form the FL adjuvant molecule (designated as FL-GPI) using a pcDNA3-GPt cassette (GenBank access number x52645), which was previously used to produce GM-CSF in an active membrane-bound form (Poloso, N. J., et al., Mol. Immunol., 38:803-816, (2002)). GPI-anchored proteins preferentially associate with lipid rafts, which are used as sites for virus assembly (Nguyen, D. H. and J. E. Hildreth; J. Virol., 74:3265-3272, (2000)). These chimeric FL constructs can be cloned into pc/pS1 and used to produce rBVs expressing FL fusion proteins.

VEE is a member of the family Togaviridae and is typically transmitted by mosquitoes to humans or other animals, in which it causes fever and encephalitis. Following inoculation into the footpad of mice, the virus was observed to be rapidly transported to the draining lymph nodes. Recent studies have shown that dendritic cells in the lymph nodes are the primary target of VEE infection, and VEE replicon particles were observed to be localized in Langerhans cells, dendritic cells of the skin, following subcutaneous inoculation (Macdonald, G. H., and Johnston, R. E., J Virol., 74 (2), 914-22, (2000)). These investigators also showed that the targeting of VEE adjuvant molecules to DCs was dependent upon the specific amino acid sequence of the viral envelope glycoprotein E2. Therefore, VLPs incorporating VEE adjuvant molecules may be used to target dendritic cells.

Dendritic cells use the mannose receptor (MR) as the major receptor for endocytosis of antigens (Sallusto, F., et al., J. Exp. Med., 192 (2), 389-400, (1995)). This receptor is a 175 kD protein containing eight carbohydrate recognition domains with high affinity for mannose-rich glycoproteins (Stahl, P. D., Curr Opin Immunol., 4 (1), 49-52, (1992) and Ezekowitz, R. A., et al., J Exp. Med., 172 (6), 1785-94, (1990)). Following endocytosis, the MR releases its ligand at low pH and it recycles to the cell surface, thus having the capacity to interact with ligands in multiple rounds (Stahl, P., et al.; Cell, 19 (1), 207-15, (1980)). It has been suggested that the MR may provide a mechanism for distinguishing self from non-self antigens on the basis of glycosylation patterns since, in higher eukaryotes, mannose residues are usually buried within the carbohydrate moieties of evelope glycoproteins and therefore not available for binding to MR (Sallusto, F., et al., J. Exp. Med. 192 (2), 389-400, (1995)). Thus, it may be possible to target VLPs to dendritic cells on the basis of distinct oligosaccharide profiles.

Once dendritic cells take up antigens, immature dendritic cells need to differentiate into professional antigen presenting cells in response to maturation signals. As dendritic cells mature, expression of co-stimulatory molecules and MHC-peptide complexes increase and cytokines are produced (Banchereau, J. & I Steinman, R. M., Nature, 392, 245-52, (1998) and Pierre, P., Turley, et al., Nature, 388, 787-92, (1997)). Interaction between CD40 expressed on antigen presenting cells including dendritic cells and CD40L on activated Th cells is important for T cell dependent B cell activation and isotype switching (Rousset, F., et al., J. Exp. Med., 173, 705-10, (1991)). CD40 ligation with a cell line expressing CD40L activates Langerhans cell-derived dendritic cells, and induces high level expression of MHC II and accessory molecules such as CD80 and CD86 (Caux, C., et al., J. Exp. Med., 180, 1263-1272, (1994)). Cross-linking CD40 with anti-CD40 antibodies play a role in ablating the tolerogenic potential of lymphoid dendritic cells (Grohmann U., et al., J. Immunol. 166, 277-83, (2001)). It is also shown that signaling through CD40 on the antigen presenting cells can replace the requirement for “help” from CD4 Th cells in inducing CTL activities (Bennett, S. R., et al., Nature, 393, 478-480, 1993 and Schoenberger, S. P., et al., Nature, 393, 480-483, (1998)). In anti-tumor pre-clinical model studies, it is indicated that the main mediator for dendritic cell activation is CD40 receptor engagement (Ribas, A., et al., Cancer Res., 61, 8787-8793, (2001) and Ridge, J. P., et al., Nature, 393, 474-478, (1998)). These studies suggest that CD40L seem to provide important maturation signals for dendritic cells. Therefore, VLPs incorporating CD40L adjuvant molecules may be used to target dendritic cells.

Follicular dendritic cells (FDCs) play an important role in germinal centers, where antibody-forming cells are generated. Recent studies have indicated that FDCs play an important co-stimulatory role in the enhancement of antibody responses (Qin, D., et al. J. Immunol., 161, 4549-4554, (1998); Fearon, D. T. and Carroll, M. C.; Annu. Rev. Immunol., 18, 393-422, (2000); Fakher, M., et al., Eur. J. Immunol., 31, 176-185, (2001) and Tew, J. G., et al., Trends Immunol., 22, 361-367, (2001)). During HIV infection, immune complexes containing virions are found in association with FDCs (Hlavacek, W. S., et al.; Philos. Trans. R. Soc. Lond B Biol. Sci., 355, 1051-1058, (2000); Rosenberg, Y. J., et al., Dev. Immunol., 6, 61-70, (1998); Smith, B. A. et al.; J. Immunol., 166, 690-696, (2001)), and such complexes could play a significant role in effective antigen presentation to B cells for induction of neutralizing antibody as observed during HIV infection in vivo. Because of their close similarity to virions, VLPs may mimic such immune complexes much more closely than soluble antigens.

The FDCs interact with components of the complement system including C3d, and it was recently demonstrated that recombinant proteins containing a segment of the C3d adjuvant molecule fused (amino acids 1024 to 1320 of SEQ ID NO: 11) to an antigen resulted in a striking increase in the efficiency of the antibody response (Dempsey, P. W., et al., Science, 271, 348-350, (1996)). Complement is a plasma protein system of innate immunity that is activated by microorganisms in the absence of antibody (Fearon, D. T. and Austen, K. F., N. Engl. J. Med., 303, 259-263, (1980)). Upon activation, C3d fragment binds to its receptor, CR2 (CD21) which is primarily expressed on B cells and FDCs (Fearon, D. T. and Carter, R. H.; Annu. Rev. Immunol., 13:17-149, (1995)). The presence of C3d adjuvant molecules on the surfaces of the VLPs may result in their enhanced interaction with FDCs and B cells, and thus stimulation of the antibody responses to viral surface envelope glycoproteins contained in the VLP structure.

Because of the relatively large size of the C3d adjuvant molecule fragment, which is about 300 amino acids in length, two factors that may affect its function: 1) its proper exposure for interaction with CR2 on FDC; and 2) its potential interference with the proper folding of the protein antigen. Two alternative approaches can be used to incorporate the C3d fragment into VLPs in order to elucidate antibody responses against viral surface glycoproteins incorporated into the VLPs.

First, the C3d adjuvant molecule fragment (amino acids 1024 to 1320 of SEQ ID NO: 11) can be fused to the N-terminus of the selected viral surface envelope glycoprotein and the tPA signal peptide (SEQ ID NO: 9) can be introduced at the N-terminus of the viral surface envelope glycoprotein. Second,. the tPA signal peptide can be fused to the N-terminus of C3d adjuvant molecule and a membrane anchoring sequence (TM domain of viral glycoproteins, example SIV envelope TM (SEQ ID NO: 6) or the GPI-anchoring sequence (GenBank access number x52645, SEQ ID NO: 10)) can be fused to the C-terminus of the C3d adjuvant molecule.

VLPs can be produced by in vitro cell culture expression systems such as, but not limited to, recombinant baculovirus expression system (BEVS) (Yamshchikov, G. V., Ritter, G. D., Vey, M., and Compans, R. W. Virology, 214, 50-58, (1995). Assembly of SIV virus-like particles containing envelope proteins can be performed using expression systems, such as, but not limited to, a baculovirus expression system Yamshchikov, G. V., Ritter, G. D., Vey, M., and Compans, R. W., Virology, 214, 50-58, (1995)), recombinant modified vaccinia Ankara expression system (MVA) (Wyatt L S, et. al., Vaccine, 15, 1451-8, (1996)), recombinant VSV, recombinant adenovirus, and recombinant DNA expression vectors. Preferably, the VLPs are produced using recombinant BEVS and recombinant MVA expression systems.

In general, VLPs can be produced by simultaneously introducing into a cell a viral protein expression vector, a viral surface envelope surface glycoprotein expression vector, and/or an adjuvant molecule expression vector. The expressed viral protein self-assembles into a VLP that incorporates the viral surface envelope glycoprotein and/or the adjuvant molecule. The viral surface envelope surface glycoprotein and/or the adjuvant molecule are expressed on the VLP surface. Thereafter, the cell produces the VLP (e.g., chimeric and/or phenotypically mixed VLP). The cells can include, but are not limited to, insect cells (e.g., spodopera frugiperda Sf9 cells and Sf21cells) and mammalian cells (e.g., EL4 cells and HeLa cells).

In general, the viral protein expression vector can be produced by operably linking a coding sequence for a viral protein of a virus to an appropriate promoter (e.g., an early promoter, late promoter, or hybrid late/very late promoter). The viral protein expression vector can also be modified to form a viral protein expression construct. In addition, the viral surface envelope glycoprotein expression vector can be produced by operably linking a coding sequence for a viral surface envelope glycoprotein of a virus to an appropriate promoter (e.g., early promoter, late promoter, or hybrid late/very late promoter). The viral surface envelope glycoprotein expression vector can be modified to form a viral surface envelope glycoprotein expression construct. Similarly, the adjuvant molecule expression vector can be produced by operably linking a coding sequence for an adjuvant molecule to an appropriate promoter (e.g., early promoter, late promoter, or hybrid late/very late promoter). The adjuvant molecule expression vector can be modified to form an adjuvant molecule expression construct.

In the case of where the adjuvant molecule is mannose, the adjuvant molecular expression construct is not needed because the mannose molecules can be chemically added to VLPs after the VLPs are produced.

The term “host” includes humans, mammals (e.g., cats, dogs, horses, and cattle), and other living species that are in need of treatment. Hosts that are “predisposed to” condition(s) can be defined as hosts that do not exhibit overt symptoms of one or more of these conditions but that are genetically, physiologically, or otherwise at risk of developing one or more of these conditions.

The term “treat”, “treating”, and “treatment” are an approach for obtaining beneficial or desired clinical results. For purposes of embodiments of this invention, beneficial or desired clinical results include, but are not limited to, alleviation of symptoms, diminishment of extent of disease, stabilization (i.e., not worsening) of disease, preventing spread of disease, delaying or slowing of disease progression, amelioration or palliation of the disease state, and remission (partial or total) whether detectable or undetectable. In addition, “teat”, “treating”, and “treatment” can also mean prolonging survival as compared to expected survival if not receiving treatment.

The term “condition” and “conditions” denote a state of health that can be related to infection by a virus. The infections that are discussed herein are to be included as conditions that can be treated by embodiments of the present invention. “Polypeptide” refers to peptides, proteins, glycoproteins; and the like, of the present invention comprising two or more amino acids joined to each other by peptide bonds or modified peptide bonds, (i.e., peptide isosteres). “Polypeptide” refers to both short chains, commonly referred to as peptides, oligopeptides, or oligomers, and to longer chains, generally referred to as proteins. “Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

Modifications may occur anywhere in the polypeptides of the present invention, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter, et al., Meth. Enzymol., 182: 626-646, (1990), and Rattan, et al., Ann New York Acad. Sci., 663:48-62, (1992)).

“Variant” refers to polypeptides of the present invention that differ from a reference polynucleotide or polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including, but not limited to, those described in Computational Molecular Biology, Lesk, A. M., Ed., Oxford University Press, New York, (1988); Biocomputing: Informatics and Genome Projects, Smith, D. W., Ed., Academic Press, New York, (1993); Computer Analysis of Sequence Data, Part I, Griffin, A. M., and Griffin, H. G., Eds., Humana Press, New Jersey, (1994); Sequence Analysis in Molecular Biology, von Heinje, G., Academic Press, 1987; and Sequence Analysis Primer, Gribskov, M. and Devereux, J., Eds., M Stockton Press, New York, (1991); and Carillo, H., and Lipman, D., SIAM J Applied Math., 48, 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48, 443-453, (1970)) algorithm (e.g., NBLAST, and XBLAST). The default parameters are used to determine the identity for the polynucleotides and polypeptides of the present invention.

By way of example, the polypeptide sequences of the present invention may be identical to one or more of the reference sequences described above, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “substantially homologous” is used herein to denote polypeptides of the present invention having about 50%, about 60%, about 70%, about 80%, about 90%, and preferably about 95% sequence identity to the sequences discussed above. Percent sequence identity is determined by conventional methods as discussed above.

In general, homologous polypeptides of the present invention are characterized as having one or more amino acid substitutions, deletions, and/or additions. These changes are preferably of a minor nature (e.g., conservative amino acid substitutions and other substitutions that do not significantly affect the activity of the polypeptide).

In addition, embodiments of the present invention include polynucleotides that encode polypeptides having one or more “conservative amino acid substitutions” of the sequences discussed above. Conservative amino acid substitutions can be based upon the chemical properties of the amino acids. Variants can be obtained that contain one or more amino acid substitutions of the sequences discussed above, in which an alkyl amino acid is substituted for an alkyl amino acid in a polypeptide, an aromatic amino acid is substituted for an aromatic amino acid in a polypeptide, a sulfur-containing amino acid is substituted for a sulfur-containing amino acid in a polypeptide, a hydroxy-containing amino acid is substituted for a hydroxy-containing amino acid in a polypeptide, an acidic amino acid is substituted for an acidic amino acid in a polypeptide, a basic amino acid is substituted for a basic amino acid in a polypeptide, or a dibasic monocarboxylic amino acid is substituted for a dibasic monocarboxylic amino acid in a polypeptide.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions include amino acids having characteristics such as a basic pH (arginine, lysine, and histidine), an acidic pH (glutamic acid and aspartic acid), polar (glutamine and asparagine), hydrophobic (leucine, isoleucine, and valine), aromatic (phenylalanine, tryptophan, and tyrosine), and small (glycine, alanine, serine, threonine, and methionine).

Polypeptides having conservative amino acid variants can also comprise non-naturally occurring amino acid residues. Non-naturally occurring amino acids include, without limitation, trans-3-methylproline, 2-4-methanoproline, cis-4-hydroxyproline, trans-4-hydroxyproline, N-methyl-glycine, allo-threonine, methylthreonine, hydroxyethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations are carried out in a cell-free system comprising an E. coli S30 extract and commercially available enzymes and other reagents. Proteins are purified by chromatography. (Robertson, et al., J. Am. Chem. Soc., 113, 2722, (1991); Ellman, et al., Methods Enzymol., 202, 301, (1991); Chung, et al., Science, 259, 806-9, (1993); and Chung, et al., Proc. Natl. Acad. Sci. USA, 90, 10145-9, (1993)).

In a second method, translation is carried out in Xenopus oocytes by microinjection of mutated mRNA and chemically aminoacylated suppressor tRNAs (Turcatti, et al., J. Biol Chem., 271, 19991-8, (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33, 7470-6, (1994)). Naturally occurring amino acid residues can be converted to non-naturally occurring species by in vitro chemical modification. Chemical modification can be combined with site-directed mutagenesis to further expand the range of substitutions (Wynn, et al., Protein Sci., 2, 395-403, (1993)).

A “chimeric” VLP, as used herein, can be defined as a VLP having at least viral surface envelope glycoprotein incorporated into the VLP, wherein the viral protein and the viral surface envelope glycoprotein are from different viruses.

A “phenotypically mixed” VLP, as used herein, can be defined can be defined as a VLP having at least one adjuvant molecule incorporated into the VLP, wherein the viral protein and the adjuvant molecule are from different viruses.

“Expressed”, as used herein, can be defined as being a molecule disposed, or a portion of the molecule disposed, upon the surface of the VLP.

An “expression construct” is an expression vector containing a coding sequence for a recombinant protein.

The term “recombinant” when used with reference to a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under-expressed or not expressed at all.

The term “operably linked” refers to the arrangement of various nucleotide sequences relative to each other such that the elements are functionally connected to and are able to interact with each other. Such elements may include, without limitation, one or more promoters, enhancers, polyadenylation sequences, and transgenes. The nucleotide sequence elements, when properly oriented, or operably linked, act together to modulate the activity of one another, and ultimately may affect the level of expression of the transgene. The position of each element relative to other elements may be expressed in terms of the 5′ terminus and the 3′ terminus of each element, and the distance between any particular elements may be referenced by the number of intervening nucleotides, or base pairs, between the elements.

A “vector” is a genetic unit (or replicon) to which or into which other DNA segments can be incorporated to effect replication, and optionally, expression of the attached segment. Examples include plasmids, cosmids, viruses, chromosomes and minichromosomes. Particularly preferred expression vectors (for expression of an attached segment) are baculovirus vectors and modified vaccinia Ankara vectors.

A “coding sequence” is a nucleotide sequence that is transcribed into mRNA and translated into a protein, in vivo or in vitro.

“Regulatory sequences” are nucleotide sequences, which control transcription and/or translation of the coding sequences, which they flank.

“Processing sites” are described in terms of nucleotide or amino acid sequences (in context of a coding sequence or a polypeptide). A processing site in a polypeptide or nascent peptide is where proteolytic cleavage occurs, where glycosylation is incorporated or where lipid groups (such as myristoylation) occurs. Proteolytic processing sites are where proteases act.

“Virus-like particles” (VLPs) are membrane-surrounded structures comprising viral envelope proteins expressed on the VLP. In addition, adjuvant molecules can be expressed on the VLP. Further, viral core proteins are located within the membrane of the VLP. Additional components of VLPs, as known in the art, can be included within or disposed on the VLP. VLPs do not contain intact viral nucleic acids, and they are non-infectious. Desirably, there is sufficient viral surface envelope glycoprotein and/or adjuvant molecules expressed, at least in part, on the surface of the VLP so that when a VLP preparation is formulated into an immunogenic composition and administered to an animal or human, an immune response (cell-mediated or humoral) is raised.

A “truncated” viral surface envelope glycoproteins are ones which contain less than a full length cytoplasmic domain, which retains surface antigenic determinants against which an immune response is generated, preferably a protective immune response, and it retains sufficient envelope sequence for proper precursor processing and membrane insertion. The skilled artisan can produce truncated virus envelope proteins using recombinant DNA technology and virus coding sequences, which are readily available to the public.

Truncation of the viral surface envelope protein is from the carboxy terminus of the viral surface envelope glycoprotein. Contemplated are truncated viral surface envelope glycoproteins retaining from about 0% to about 90% of the cytoplasmic domain, where the cytoplasmic domain for a type I viral surface envelope glycoprotein is the region between the C-terminus of the transmembrane domain and the C-terminus of the entire envelope protein. Preferably from about 10% to 25% of the cytoplasmic domain is retained. Also encompassed by “truncated,” are all percentages and ranges, within the 5% to 90% range, including from about 15% to about 50%.

VLPs based on cloned viral surface envelope glycoproteins, and preferably further comprising Gag proteins from the same viruses, can be readily produced without the expense of undue experimentation by the ordinary skilled artisan using the teachings of the present application taken with vectors as described herein and what is well known to and readily accessible in the art.

In another embodiment, polyclonal and/or monoclonal antibodies capable of specifically binding to the VLP are provided. The term “antibody” is used to refer both to a homogenous molecular entity, or a mixture such as a serum product made up of a plurality of different molecular entities. Monoclonal or polyclonal antibodies, which specifically react with the VLPs of the present invention, may be made by methods known in the art. (e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1987)). Also, recombinant immunoglobulins may be produced by methods known in the art, including but not limited to, the methods described in U.S. Pat. No. 4,816,567.

Antibodies specific for VLPs and viral surface envelope glycoproteins of viruses may be useful, for example, as probes for screening DNA expression libraries or for detecting the presence of the cognate virus in a test sample. Frequently, the polypeptides and antibodies will be labeled by joining, either covalently or noncovalently, a substance that provides a detectable signal. Suitable labels include but are not limited to radionuclides, enzymes, substrates, cofactors, inhibitors, fluorescent agents, chemiluminescent agents, magnetic particles and the like. United States patents describing the use of such labels include but are not limited to U.S. Pat. Nos. 3,817,837; 3,850,752; 3,939,350; 3,996,345; 4,277,437; 4,275,149; and 4,366,241.

Antibodies specific for VLPs and retroviral surface envelope glycoproteins may be useful in treating animals, including humans, suffering from cognate viral disease. Such antibodies can be obtained by the methods described above and subsequently screening the viral surface envelope glycoproteins-specific antibodies for their ability to inhibit virus uptake by target cells.

Compositions and immunogenic preparations of the present invention, including vaccine compositions, comprising the VLPs of the present invention and capable of inducing protective immunity in a suitably treated host and a suitable carrier therefor are provided. “Immunogenic compositions” are those which result in specific antibody production or in cellular immunity when injected into a host. Such immunogenic compositions or vaccines are useful, for example, in immunizing hosts against infection and/or damage caused by viruses, including, but not limited to, HIV, human T-cell leukemia virus (HTLV) type I, SIV, FIV, bovine immunodeficiency virus, bovine leukemia virus and equine infectious anemia virus, SARS, RVFV, Filovirus, Flavivirus, Arena virus, and Bunya virus.

The vaccine preparations of the present invention can include an immunogenic amount of one or more VLPs, fragment(s), or subunit(s) thereof. Such vaccines can include one or more viral surface envelope glycoproteins and portions thereof, and adjuvant molecule and portions thereof on the surfaces of the VLPs, or in combination with another protein or other immunogen, such as one or more additional virus components naturally associated with viral particles or an epitopic peptide derived therefrom.

By “immunogenic amount” is meant an amount capable of eliciting the production of antibodies directed against the virus, in the host to which the vaccine has been administered. It is preferred for SIV, HIV and HTLV, among others, that the route of administration and the immunogenic composition is designed to optimize the immune response on mucosal surfaces, for example, using nasal administration (via an aerosol) of the immunogenic composition.

Immunogenic carriers can be used to enhance the immunogenicity of the VLPs from any of the viruses discussed herein. Such carriers include, but are not limited to, proteins and polysaccharides, microspheres formulated using (e.g., a biodegradable polymer such as DL-lactide-coglycolide, liposomes, and bacterial cells and membranes). Protein carriers may be joined to the proteinases or peptides derived therefrom to form fusion proteins by recombinant or synthetic means or by chemical coupling. Useful carriers and means of coupling such carriers to polypeptide antigens are known in the art.

The immunogenic compositions and/or vaccines of the present invention may be formulated by any of the means known in the art. They can be typically prepared as injectables or as formulations for intranasal administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active immunogenic ingredients are often mixed with excipients or carriers, which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable, aerosol or nasal formulations is usually in the range of 0.2 to 5 mg/ml. Similar dosages can be administered to other mucosal surfaces.

In addition, if desired, the vaccines may contain minor amounts of auxiliary substances such as wetting or emulsifying agents, pH buffering agents, and/or other agents, which enhance the effectiveness of the vaccine. Examples of agents which may be effective include, but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of the auxiliary substances may be determined by measuring the amount of antibodies (especially IgG, IgM or IgA) directed against the immunogen resulting from administration of the immunogen in vaccines which comprise the adjuvant in question. Additional formulations and modes of administration may also be used.

“Pharmaceutically acceptable salts” include, but are not limited to, the acid addition salts (formed with free amino groups of the peptide) which are formed with inorganic acids (e.g., hydrochloric acid or phosphoric acids) and organic acids (e.g., acetic, oxalic, tartaric, or maleic acid). Salts formed with the free carboxyl groups may also be derived from inorganic bases (e.g., sodium, potassium, ammonium, calcium, or ferric hydroxides), and organic bases (e.g., isopropylamine, trimethylamine, 2-ethylamino-ethanol, histidine, and procaine).

The immunogenic compositions and/or vaccines of the present invention can be administered in a manner compatible with the dosage formulation, and in such amount and manner as will be prophylactically and/or therapeutically effective, according to what is known to the art. The quantity to be administered, which is generally in the range of about 1 to 1,000 micrograms of viral surface envelope glycoprotein per dose and/or adjuvant molecule per dose, more generally in the range of about 5 to 500 micrograms of glycoprotein per dose and/or adjuvant molecule per dose, depends on the subject to be treated, the capacity of the hosts immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

The vaccine or immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response (e.g., at 1 to 4 months for a second dose, and if needed, a subsequent dose(s) after several months). Humans (or other animals) immunized with the VLPs of the present invention are protected from infection by the cognate virus.

It should also be noted that the vaccine or immunogenic composition can be used to boost the immunization of a host having been previously treated with a vaccine such as, but not limited to, DNA vaccine and a recombinant virus vaccine.

Except as noted hereafter, standard techniques for peptide synthesis, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y., Old Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, N.Y.

Abbreviations and nomenclature, where employed, are deemed standard in the field and commonly used in professional journals such as those cited herein.

All publications, including, but not limited to, patents, patent applications, and papers, cited in this specification are herein incorporated by reference as if each individual publication were specifically and individually indicated to be incorporated by reference herein as though fully set forth.

It should be emphasized that the above-described embodiments of the present invention are merely possible examples of implementations, and are set forth only for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A virus-like particle (VLP), comprising:
a viral core protein that can self assemble into the VLP core; and
at least one viral surface envelope glycoprotein expressed on the surface of the VLP, wherein the viral protein and the viral surface envelope glycoprotein are from different viruses.

2. The VLP of claim 2, wherein the viral core protein is selected from a viral Gag viral core protein, an HIV Gag protein, an SIV Gag protein, an MuLV Gag viral core protein, a VSV M viral core protein, an Ebola VP40 viral core protein, corona virus M and E proteins, a Bunyavirus N Protein viral core protein, and combinations thereof.

3. The VLP of claim 2, wherein the viral surface envelope surface glycoprotein is selected from a human immunodeficiency virus (HIV), a feline immunodeficiency virus (FIV), a feline leukemia virus, a bovine immunodeficiency virus, a bovine leukemia virus, a equine infectious anemia virus, a human T-cell leukemia virus, a Bunyavirus glycoprotein, a Lassa fever virus glycoprotein, an Ebola virus glycoprotein, a corona virus glycoprotein, an arenavirus glycoprotein, a Filovirus glycoprotein, an influenza virus glycoprotein, a paramyxovirus glycoprotein, a rhabdovirus glycoprotein, an alphavirus glycoprotein, a flavivirus glycoprotein, and combinations thereof.

4. A virus-like particle (VLP), comprising:
a viral core protein that can self assemble into a VLP core;
at least one viral surface envelope glycoprotein expressed on the surface of the VLP; and
at least one adjuvant molecule expressed on the surface of the VLP.

5. The VLP of claim 4, wherein the viral core protein and the viral surface envelope glycoprotein are from different viruses.

6. The VLP of claim 4, wherein the viral core protein and the viral surface envelope glycoprotein are from the same virus.

7. The VLP of claim 4, wherein the viral core protein is selected from an HIV Gag viral core protein, an SIV Gag viral core protein, an MuLV Gag viral core protein, a VSV M viral core protein, an Ebola VP40 viral core protein, a corona virus M protein, a corona virus E protein, a Bunya Virus N Protein viral core protein, and combinations thereof.

8. The VLP of claim 4, wherein the viral surface envelope surface glycoprotein is selected from a human immunodeficiency virus (HIV), a simian-human immunodeficiency virus (SHIV), a feline immunodeficiency virus (FIV), a feline leukemia virus, a bovine immunodeficiency virus, a bovine leukemia virus, a equine infectious anemia virus, a human T-cell leukemia virus, a Bunya Virus glycoprotein, a Lassa fever virus glycoprotein, an Ebola virus glycoprotein, corona virus glycoprotein, an Arena virus glycoprotein, a Filo viruse glycoprotein, an influenza virus glycoprotein, a paramyxovirus glycoprotein, a rhabdo virus glycoprotein, an alphavirus glycoprotein, a flavi virus glycoprotein, and combinations thereof.

9. The VLP of claim 4, wherein the at least one adjuvant molecule is selected from an influenza HA adjuvant molecule, a parainfluenza HN adjuvant molecule, a Venezuelan equine encephalitis (VEE) adjuvant molecule, a F1t3 adjuvant molecule, a C3d adjuvant molecule, a mannose receptor adjuvant molecule, a CD40 adjuvant molecule, and combinations thereof.

10. An immunogenic composition, comprising the VLP of claim 1 and a pharmacologically acceptable carrier.

11. An immunogenic composition, comprising the VLP of claim 4 and a pharmacologically acceptable carrier.

12. A method of generating an immunological response in a host, comprising the step of administering an effective amount of the immunogenic composition of claim 10 to the host.

13. A method of generating an immunological response in a host, comprising the step of administering an effective amount of the immunogenic composition of claim 11 to the host.

14. A method of treating a condition, comprising administering to a host in need of treatment an effective amount of the immunogenic composition of claim 10.

15. A method of treating a condition, comprising administering to a host in need of treatment an effective amount of the immunogenic composition of claim 11.

16. A method of determining exposure of a host to a virus, said method comprising the steps of:
contacting a biological fluid of the host with a virus-like particle (VLP) selected from the VLP of claim 1, the VLP of claim 4, and combinations thereof, wherein the VLP is of the same virus type to which exposure is being determined, under conditions which are permissive for binding of antibodies in the biological fluid with the VLP; and
detecting binding of antibodies within the biological fluid with the VLP, whereby exposure of the host to the virus is determined by the detection of antibodies bound to the VLP.

17. The method of claim 16, wherein detecting includes the use of a labeled second antibody which is specific to antibodies in the biological fluid being tested.

18. A method for targeting antigen presenting cells in a host, comprising the step of administering an effective amount of the immunogenic composition of claim 11 to the host, wherein the at least one adjuvant molecule is selected from a Venezuelan equine encephalitis (VEE) adjuvant molecule, a F1t3 adjuvant molecule, a C3d adjuvant molecule, a mannose receptor adjuvant molecule, a CD40 adjuvant molecule, and combinations thereof.

19. The method for targeting antigen presenting cells in a host of claim 18, wherein the antigen presenting cell is a dendritic cell and the at least one adjuvant molecule is selected from a Venezuelan equine encephalitis (VEE) adjuvant molecule, a F1t3 adjuvant molecule, a mannose receptor adjuvant molecule, a CD40 adjuvant molecule, and combinations thereof.

20. The method for targeting antigen presenting cells in a host of claim 14, wherein the antigen presenting cell is a follicular dendritic cell and the at least one adjuvant molecule is a C3d adjuvant molecule.

21. A method for targeting mucosal surfaces in a host, comprising the step of administering an effective amount of the immunogenic composition of claim 11 to the host, wherein the at least one adjuvant molecule is selected from an influenza HA adjuvant molecule, a parainfluenza HN adjuvant molecule, and combinations thereof.

22. A method for producing humoral and cellular immune responses in mucosal and systemic compartments, comprising administering an effective amount of the immunogenic composition of claim 11 to the host, wherein the at least one viral surface envelope surface glycoprotein is selected from human immunodeficiency virus (HIV), and wherein the at least one adjuvant molecule is selected from an influenza HA adjuvant molecule, a parainfluenza HN adjuvant molecule, and combinations thereof.

23. A method of making a virus-like particle (VLP), comprising the steps of:
providing a viral core protein expression vector;
providing a viral surface envelope surface glycoprotein expression vector;
providing a adjuvant molecule expression vector; and
introducing into a cell the viral core protein expression vector, the viral surface envelope surface glycoprotein expression vector, and the adjuvant molecule expression vector and allowing for expression of the viral surface envelope surface glycoprotein and the adjuvant molecule,
whereby the VLP is formed by the cells.

24. The method of claim 23, wherein the VLP is selected from human immunodeficiency virus (HIV) VLP, a simian-human immunodeficiency virus (SHIV) VLP, a feline immunodeficiency virus (FIV) VLP, a feline leukemia virus VLP, a bovine immunodeficiency virus VLP, a bovine leukemia virus VLP, a equine infectious anemia virus VLP, a human T-cell leukemia virus VLP, a Bunya Virus VLP, Lassa fever virus VLP, Ebola virus VLP, corona virus VLP, Arena virus VLP, Filovirus VLP, influenza virus VLP, paramyxovirus VLP, rhabdo virus VLP, alphavirus VLP, and flavi virus VLP.

25. A method of making a virus-like particle (VLP), comprising the steps of:
operably linking a coding sequence for a viral core protein of a virus to a promoter, and inserting it into a viral core protein expression vector;
operably linking a coding sequence for a viral surface envelope glycoprotein of a virus to a promoter, and inserting it into a viral surface envelope surface glycoprotein expression vector;
operably linking a coding sequence for an adjuvant molecule to a promoter, and inserting it into a adjuvant molecule expression vector;
simultaneously introducing into a cell the viral core protein expression vector, the viral surface envelope surface glycoprotein expression vector, and the adjuvant molecule expression vector and allowing for expression of the viral surface envelope surface glycoprotein and the adjuvant molecule,
whereby the VLP is formed by the cells.

26. The method of claim 25, wherein the promoter is selected from a baculovirus promoter and a recombinant modified vaccinia Ankara (MVA) promoter, a CMV promoter, EF promoter, adenovirus promoter, recombinant VSV, recombinant adenovirus, recombinant alphavirus, and recombinant DNA expression vectors.

27. A method of boosting the immunization of a host having been previously treated with a vaccine, comprising the step of administering an effective amount of the immunogenic composition of claim 10 to the host.

28. A method of boosting the immunization of a host having been previously treated with a vaccine, comprising the step of administering an effective amount of the immunogenic composition of claim 11 to the host.


SEQUENCE LISTING (11 sequences)

SEQ ID NO: 1 - protein, 847 aa, HIV SF162 Envelope Protein
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIKIFIMIVGGLVGLRIVFTVLSIVNRVRQGYSPLSFQTRFPAPRGPDRPEGIEEEGGERDRDRSSPLVHGLLALIWDDLRSLCLFSYHRLRDLILIAARIVELLGRRGWEALKYWGNLLQYWIQELKNSAVSLFDAIAIAVAEGTDRIIEVAQRIGRAFLHIPRRIRQGFERALL

SEQ ID NO: 2 - protein, 1067 aa, RVFV Glycoprotein a.a.
MAGIAMTVLPALAVFALAPVVFAEDPHLRNRPGKGHNYIDGMTQEDATCKPVTYAGACSSFDVLLEKGKFPLFQSYAHHRTLLEAVHDTIIAKADPPSCDLQSAHGNPCMKEKLVMKTHCPNDYQSAHYLNNDGKMASVKCPPKYGLTEDCNFCRQMTGASLKKGSYPLQDLFCQSSEDDGSKLKTKMKGVCEVGVQAHKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHGCIVCAHGLINYQCHTALSAFVVVFVFSSIAIICLAVLYRVLKCLKIAPRKVLNPLMWITAFIRWIYKKMVARVAHNINQVNREIGWMEGGQLVLGNPAPIPRHAPIPRYSTYLMLLLIVSYASACSELIQASSRITTCSTEGVNTKCRLSGTALIRAGSVGAEACLMLKGVKEDQTKFLKIKTVSSELSCREGQSYWTGSISPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPSKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGRTGLSKMWLAATKKAS

SEQ ID NO: 3 - protein, 853 aa, HIV 89.6 Envelope Glycoprotein
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLWNWFDITNWLWYIRLFIMIVGGLIGLRIVFAVLSIVNRVRQGYSPLSFQTLLPASRGPDRPEGTEEEGGERDRDRSGPLVNGFLALFWVDLRNLCLFLYHLLRNLLLIVTRIVELLGRRGWEALKYWWNLLQYWSQELKNSAVSLLNATAIAVAEGTDRVIKIVQRACRAIRNIPTRIRQGLERALL

SEQ ID NO: 4 - protein, 507 aa, RVFV GC
CSELIQASSRITTCSTEGVNTKCRLSGTALIRAGSVGAEACLMLKGVKEDQTKFLKIKTVSSELSCREGQSYWTGSISPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPSKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGRTGLSKMWLAATKKAS

SEQ ID NO: 5 - protein, 527 aa, RVFV GN
EDPHLRNRPGKGHNYIDGMTQEDATCKPVTYAGACSSFDVLLEKGKFPLFQSYAHHRTLLEAVHDTIIAKADPPSCDLQSAHGNPCMKEKLVMKTHCPNDYQSAHYLNNDGKMASVKCPPKYGLTEDCNFCRQMTGASLKKGSYPLQDLFCQSSEDDGSKLKTKMKGVCEVGVQAHKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHGCIVCAHGLINYQCHTALSAFVVVFVFSSIAIICLAVLYRVLKCLKIAPRKVLNPLMWITAFIRWIYKKMVARVAHNINQVNREIGWMEGGQLVLGNPAPIPRHAPIPRYSTYLM

SEQ ID NO: 6 - protein, 26 aa, N-terminus of the SIV Env Glyocoprotein-41 TM domain
YIQYGVYIVVGVILLRIVIYIVQMLA

SEQ ID NO: 7 - protein, 232 aa, extracellular coding domain of the FL gene
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 8 - protein, 164 aa, SIV mac239 Env cytoplasmic domain
KLRQGYRPVFSSPPSYFQQTHIQQDPALPTREGKERDGGEGGGNSSWPWQIEYIHFLIRQLIRLLTWLFSNCRTLLSRVYQILQPILQRLSATLQRIREVLRTELTYLQYGWSYFHEAVQAVWRSATETLAGAWGDLWETLRRGGRWILAIPRRIRQGLELTLL

SEQ ID NO: 9 - protein, 23 aa, tPA signal peptide
MDAMKRGLCCVLLLCGAVFVSAR

SEQ ID NO: 10 - protein, 40 aa, GPI-anchoring seq. access no. x52645
LAVSTISSFFPPGYQVSFCLVMVLLFAVDTGLYFSVKTNI

SEQ ID NO: 11 - protein, 300 aa, C3d sequence
TVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRSSATTFRLLWENGNLLR